(19) United States
(12) Patent Application Publication
(10) Pub. No.: US 2005/0089558 A1
(43) Pub. Date: Apr. 28, 2005

(54) Compositions and methods for the co-formulation and administration of tramadol and propoxyphene

(75) Inventor: Neal R. Cutler, Los Angeles, CA (US)

Correspondence Address: Thomas W. Brown;MELDEN & CARROLL, LLP, Suite 350, 101 Howard Street, San Francisco, CA, 94105, US

(73) Assignees: Alamo Pharmaceuticals, LLC; R.T. Alamo Ventures III, LLC, Beverly Hills, CA (US)

(21) Appl. No.: 10/695,064

(22) Filed: Oct. 28, 2003

(51) Int. Cl. A61K 9/20; A61K 9/22; A61K 31/5377

(52) U.S. Cl. 424/464; 514/235.5

(57) ABSTRACT

Methods and pharmaceutical compositions for treating subjects having pain are provided. The methods of the present invention comprise the utilization of combined pharmaceutical formulations comprising the tramadol and propoxyphene in subjects suffering from pain.

### FIELD OF THE INVENTION

The present invention relates to pharmaceutical compositions and methods for the treatment of pain in subjects in need of relief from pain.

### BACKGROUND

There are more than 50 million Americans who experience chronic pain. Pain is a frequent cause for clinical visits, with approximately 45% of the population seeking medical help for pain at some point in their lives. More than half of dying patients experience moderate to severe pain during the last days of their life.

Pain is frequently undertreated by healthcare providers. It has been estimated that four out of every ten people with moderate or severe pain do not get adequate relief. For example, a survey of several hundred ambulatory AIDS patients found that fewer than 8% of patients reporting “severe” pain were prescribed a strong opioid such as morphine, despite published guidelines. Adjuvant analgesic drugs (e.g., antidepressants) were also prescribed to only a small fraction of these patients.

Opioid analgesics are the accepted treatment for acute pain, cancer pain, and pain at the end of life. Recently, opioid analgesics have been recommended for chronic, nonmalignant pain. However, many health care providers are loath to prescribe effective doses of opioid analgesics for fear that sustained use, at therapeutic doses, may result in a downhill spiral of further disability, depression, and pain.

In addition to health care provider barriers, there are patient and family barriers to effective pain relief. Patients may underuse effective pharmacological treatments because of a stoic or fatalistic attitude, and/or a belief that complaining of pain makes one a “bad” patient. Patients who are treated with opioids may have additional fears of dependence, addiction and tolerance. What is needed, therefore, are modalities for the treatment of pain in subjects in need of relief from pain.

### SUMMARY OF THE INVENTION

The present invention provides compositions and methods for the treatment of pain. In one embodiment, a method of treating a patient comprising the administration of tramadol with propoxyphene in a combined formulation (i.e. a single dosage form with both drugs) or combined administration (e.g. the co-administration of discrete dosage forms of each drug). Such combined formulations or administration permit both drugs to be used at a lower dose than that used for each drug in monotherapy (i.e. when the drugs are given alone). For example, in one embodiment, it is contemplated that the dose of each drug in the combined formulation or administration may be between 0.1 and 0.75 of the upper dose used in monotherapy, preferably between 0.25 and 0.75 of the upper dose used in monotherapy. Such combinations may also permit a better treatment profile for the subject. For example, in one embodiment, a method of treating a subject with a combined formulation or administration comprising tramadol and propoxyphene is contemplated, such that onset of analgesia (from the time the drugs are administered) is shortened and the duration of analgesia is increased. In another embodiment, at least one of the factors contributing to “breakthrough pain” is improved through the combined administration of tramadol and propoxyphene.

In one embodiment, the treatment of a subject who has previously been unsuccessfully treated with tramadol monotherapy is contemplated. Unsuccessful treatment with tramadol may present in various ways, including, but not limited to an unacceptable degree of analgesia and toxicities associated with increased doses of tramadol. This tramadol toxicity is characterized by neurologic toxicity such as seizures, coma and respiratory depression, while cardiovascular toxicity is characterized by tachycardia and hypertension. In such subjects, the treatment with a combined formulation or administration comprising tramadol and propoxyphene is anticipated to result in more successful treatment than treatment with tramadol alone. By more successful treatment, it is contemplated that side-effects (associated with the administration of tramadol or propoxyphene alone) will be reduced. Side-effects of tramadol administration are “reduced” when the magnitude (e.g. intensity) or frequency of symptom(s), associated with the side effect, is reduced. For example the cardiovascular toxicities, associated with some dosages of tramadol monotherapy, are reduced when a subject's tachycardia is decreases toward a normal heart rate and/or a subject's hypertension is reduced toward nonmotensive levels. It is not intended that the present invention be limited only to cases where these side effects are eliminated. The present invention specifically contemplates treatment such that these side effects are reduced (and the condition of the patient is thereby “improved”), albeit not completely eliminated.

In one embodiment, the treatment of a subject who has previously been unsuccessfully treated with propoxyphene monotherapy is contemplated. Unsuccessful treatment with propoxyphene may present in various ways, including, but not limited to inadequate analgesia at low doses (i.e. 30 mg or less once every four to six hours) and toxicities at high doses (i.e. 100 mg once every four to six hours) which could include: amnesia, cognitive dysfunction, hallucinations, seizure, serotonin disorders, syncope, orthostatic hypotension and tachycardia. In such subjects, the treatment with a combined formulation or administration comprising tramadol and propoxyphene is anticipated to result in more successful treatment than treatment with propoxyphene alone. By more successful treatment, it is contemplated that the efficacy is improved and side-effects (associated with the sole administration of propoxyphene) will be reduced. Side-effects of propoxyphene administration are “reduced” when the magnitude (e.g. intensity) or frequency of symptom(s), associated with the side effect, is reduced. It is not intended that the present invention be limited only to cases where these side effects are eliminated. The present invention specifically contemplates treatment such that these side effects are reduced (and the condition of the patient is thereby “improved”), albeit not completely eliminated.

In one embodiment, the dose of one of the two active compounds (i.e. tramadol and propoxyphene) in the combined formulation or administration is reduced to between one-tenth and one-half of the upper dose at which it is normally prescribed, while the dose of the other active compound is the same as the dose at which it is normally prescribed. Such formulations, with markedly reduced dosages of each active compound, are anticipated to be particularly useful in the treatment of elderly subjects (i.e. subjects generally over 60 years of age, and more typically over 65 years of age). Treatment of non-elderly subjects is also contemplated with such formulations or combined administration.

In another preferred embodiment, the dose of each of tramadol and propoxyphene will be approximately one-half the usual upper dose of each drug when used in monotherapy. For example, 50 mg tramadol formulated or administered in combination with 30 mg propoxyphene. In another embodiment, the dose of one drug (i.e. tramadol or propoxyphene) is one quarter the normal upper dose, and the dose of the other drug is three quarters the normal upper dose.

In one embodiment, the combined formulation comprising tramadol and propoxyphene will be administered orally. In another embodiment the combined formulation comprising tramadol and propoxyphene will be suitable for sublingual or buccal administration. In some embodiments, the combined formulations suitable for oral, buccal or sublingual administration will be rapidly dissolving. In yet another embodiment, the combined formulation comprising tramadol and propoxyphene will be administered transdermally (i.e. as a formulation suitable for absorption through the skin, such as in skin patches). In another embodiment, the combined formulation comprising tramadol and propoxyphene will be administered by injection (i.e. subcutaneously, intramuscularly or intravenously). In another embodiment, the combined formulation comprising tramadol and propoxyphene will be administered intranasally. The present invention also relates to such compositions, as well as their administration to subjects. Such compositions will further comprise pharmaceutically acceptable excipients, buffers, stabilizers and additional non-active agents or inert ingredients as necessary.

In one embodiment, the present invention contemplates a method for the treatment of pain comprising, providing: a subject with pain; a combined formulation comprising tramadol and propoxyphene; and, administering said combined formulation to said subject such that said pain is reduced.

In one embodiment, said subject has been previously treated with tramadol monotherapy.

In one embodiment, a combined formulation of tramadol and propoxyphene is formulated as a tablet suitable for oral administration.

In another embodiment, a combined formulation of tramadol and propoxyphene is formulated as a tablet further comprising a controlled release solid dosage form suitable for oral administration.

In one embodiment, the controlled release solid dosage form further comprises an initial rapid release component.

In one embodiment, a combined formulation of tramadol and propoxyphene is formulated as tablet further comprising a rapidly dissolving solid dosage form suitable for oral administration.

In one embodiment, a combined formulation of tramadol and propoxyphene is formulated as a sublingual dosage form.

In one embodiment, a combined formulation of tramadol and propoxyphene is formulated as an intranasal dosage form.

Definitions

As used herein, “tramadol” refers to the compound with the following systematic names: cyclohexanol, 2-((dimethylamino)-1-(3-methoxyphenyl)-, cis-(+−)- and cyclohexanol, 2-((dimethylamino)methyl)-1-(3-methoxyphenyl)-, cis-(+−)- and also includes all esters and salts (including but not limited to tramadol hydrochloride) thereof. Tramadol has a molecular formula of C₁₆H₂₅NO₂ and the following chemical structure:

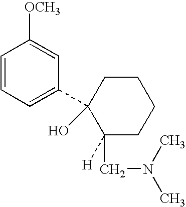

As used herein, “propoxyphene” refers to the compound with the following systematic names: 2-Butanol, 4-(dimethylamino)-3-methyl-1,2-diphenyl-, propionate, (+)-; Benzeneethanol, alpha-((1R)-2-(dimethylamino)-1-methylethyl)-alpha-phenyl-, propanoate (ester), (alphaS)-; Benzeneethanol, alpha-(2-(dimethylamino)-1-methylethyl)-alpha-phenyl-, propanoate (ester), (S—(R*,S*))- and also includes and also includes the individual enantiomers as well as all esters and salts (including but not limited to dextro-propoxyphene hydrochloride). Propoxyphene has a molecular formula of C₂₂H₂₉NO₂ and the following chemical structure:

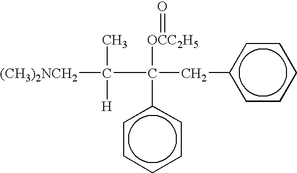

As used herein, “drug half-life” refers to the time it takes for half of a given amount of drug to be eliminated from the body (e.g. as determined by measurements of serum and/or urine drug levels).

As used herein, “subject” refers to both humans and animals.

As used herein, “patient” refers to a person receiving medical treatment.

As used herein, “granule” refers to a small grain or pellet.

As used herein, “elderly subject” refers to a human subject, generally over 60 years of age, and more typically over 65 years of age.

As used herein, “treatment” refers to a reduction of symptoms or to a reduction of side effects. Symptoms are “reduced” when the magnitude (e.g. intensity) or frequency of symptoms is reduced. In the case of analgesia, symptoms are reduced when the sensation of pain is diminished. It is not intended that the present invention is limited to any specific type of pain. For example the treatment of orthopedic, muscular, abdominal, urological, and gynecological pain is expressly contemplated. In addition, the treatment of headache (especially migraine headache) is also contemplated. The treatment of “breakthrough pain” is also contemplated. The present invention specifically contemplates treatment such that one or more symptoms are reduced (and the condition of the subject is thereby “improved”), albeit not completely eliminated. The present invention is also not limited to the reduction of all pain. In one embodiment, muscle pain (for example) is reduced whereas the patients other pain is not reduced.

As used herein, “combined formulation” refers to the mixture of two or more isolated pharmaceutical compositions comprising (tramadol and propoxyphene in one example) into a single dosage form.

As used herein, “combined administration” or “co-administration” refers to administration of two or more isolated pharmaceutical compositions (tramadol and propoxyphene in one example) in separate dosage forms (e.g. separate pills) taken together.

As used herein, “single dosage” refers to a pharmaceutical composition of a formulation that is capable of achieving its intended effect in a single application or administration.

As used herein, “oral administration” or “orally” refers to the introduction of a pharmaceutical composition into a subject by way of the oral cavity (e.g., in liquid or solid form).

As used herein, “sublingual administration” or “sublingually” refers to the introduction of a pharmaceutical composition into a subject by application to the mucosal surface under the tongue (within the oral cavity) such that the composition is absorbed into the subject.

As used herein, “buccal administration” or “buccal” refers to the introduction of a pharmaceutical composition into a subject by application to the mucosal surface lining the cheek (within the oral cavity) such that the composition is disintegrated, dissolved and absorbed into the subject. In some instances, disintegration and dissolution occurs in the buccal cavity, followed by absorption of all or a portion of the pharmaceutically active ingredient through the buccal mucosa. The remaining pharmaceutically active ingredient, if any, is then swallowed and absorbed enterally. As used herein, “intranasal administration” or “intranasally” refers to the introduction of a pharmaceutical composition within the nasal cavity.

As used herein, “respiratory inhalation” refers to the introduction of a pharmaceutical composition within the respiratory tract.

As used herein, “transdermal administration” or “transdermally” or “cutaneously” refers to the introduction of a pharmaceutical composition into a subject by application to the surface of the skin such that the composition is absorbed into the subject.

As used herein, “sustained-release” and “controlled-release” refers to a formulation wherein the active ingredient(s), in one example tramadol and/or propoxyphene, in a formulation are released over a length of time. More specifically, the active ingredients in the formulation are released over a period of about one to twelve hours. In many cases, therefore, sustained release formulations provide sustained therapeutic effect(s) with only one or two administrations, of a given dosage form, per day.

As used herein, “injection” or “standard injection” refers to the placement of a pharmaceutical composition into a subject (e.g., with a hypodermic needle). For example, such injection can be made subcutaneously, intravenously, intramuscularly, intracavemosally, etc.

As used herein, “tolerance” refers to the state or ability of being less responsive to a drug or stimulus, especially over a period of continued exposure. Tolerance can be manifested by a decreased effect in response to a given dose of the drug, or when increasingly larger doses must be taken to obtain the effects observed with the original dose.

As used herein, “breakthrough pain” refers to intermittent flares of pain that can occur even though a person is taking analgesic medications on a fixed schedule for pain control. These flares of pain are called “breakthrough pain” because the pain “breaks through” the regular pain medication. About one-half to two thirds of patients with chronic cancer-related pain also experience episodes of breakthrough cancer pain (see, Portenoy R K and Hagen N A, Pain 1990;41:273-281). The characteristics of breakthrough cancer pain vary from person to person, including the duration of the breakthrough episode and possible causes.

As used herein, the abbreviation “ED” refers to “Effective Dose.” The effective dose is the dose (of a drug) that produces the desired effect such that when followed by a subscript (i.e.“ED_{yy}”), it denotes the dose having such an effect on a certain percentage (e.g., yy %) of the test animals. By way of example, which is not intended to limit the dosages of the described in the present invention; “ED₅₀” refers to the dose of a drug experimentally found or predicted (by statistical techniques) to produce a characteristic effect in 50 percent of the subjects to whom the dose is given. The median effective dose (abbreviated ED₅₀) is found by interpolation from a dose-effect curve. The ED₅₀ is readily used as a standardized dose by means of which the potencies of drugs may be compared. However, standardized doses may also be calculated using values between ED₁ and ED₉₉.

As used herein a “writhing-induction agent” is an agent that, upon intraperitoneal administration, irritates the serous membranes and provokes a very stereotyped behavior in the mouse and rat. This stereotyped behavior is characterized by abdominal contractions, movement of the body as a whole (particularly of the hind paws), twisting of dorso-abdominal muscles, and a reduction in motor activity and motor coordination.

As used herein a “rapidly dissolving solid dosage form” refers to a dosage formulated such that no less than 85% of the propoxyphene and tramadol, combined therein, dissolves within 1-2 minutes of administration.

As used herein an “initial rapid release component” refers to a dosage formulated such that, upon ingestion, the initial rapid release component of the dosage form rapidly disintegrates such that approximately 30% of the tramadol and propoxyphene contained, within the dosage form, is released for rapid systemic absorption.

### DESCRIPTION OF THE INVENTION

The present invention relates to methods and compositions useful in the treatment of pain. In particular, the present invention contemplates combined formulations comprising tramadol and propoxyphene and methods of administering such combined formulations to subjects in order to treat pain. It is not intended that the present invention be limited to the treatment of any specific type of pain. However, the compositions and methods described by certain embodiment of the present invention are expected to be therapeutic in the treatment of the following categories of pain.

I. Examples of Types of Pain that may be Treated by Embodiments of the Present Invention

A. Acute Pain

Acute pain has been defined as lasting less than 6 weeks and is related to a discernible incident such as surgery or trauma. Moreover acute pain, usually while traditionally seen as temporary, is now envisioned as the initiation phase of an extensive, persistent nociceptive and behavioural cascade of reactions triggered by tissue injury. Within minutes of trauma, phenotypic changes are observed in primary afferent as well as spinal cord and brain nociceptive neurons, and these changes are the basis for long-term sensitization. It is contemplated that the co-administration of tramadol and propoxyphene or the administration of a tramadol and propoxyphene co-formulation will be especially well suited to the treatment of acute pain. In a preferred embodiment, it is contemplated that the co-administration of tramadol and propoxyphene or the administration of a tramadol and propoxyphene co-formulation will be effective in the treatment of acute post-operative pain in adult surgical patients, especially patients who have undergone coronary bypass artery graft surgery.

B. Chronic Non-Cancer Pain

Chronic pain has been defined as pain lasting more than 6 weeks and related to an ongoing pathophysiology. Chronic non-cancer pain has been subdivided into the following categories.

i. Somatic Pain

Somatic pain arises in skin, bone, and muscle. Examples are bone and joint pain resulting from injury, rheumatoid arthritis, osteoarthritis, sickle cell anemia, or chronic osteomyelitis; chronic headache; and chronic back pain related to injury or multiple surgeries.

ii. Visceral Pain

Visceral pain involves the visceral organs. Examples are chronic pelvic pain and chronic interstitial cystitis.

iii. Neuropathic Pain

Neuropathic pain—results from injury to nerves. Examples are peripheral diabetic neuropathy, reflex sympathetic dystrophy, and post-herpetic neuralgia. It is contemplated that the co-administration of tramadol and propoxyphene or the administration of a tramadol and propoxyphene co-formulation will be especially well suited to the treatment of chronic non-cancer pain.

C. Cancer Pain

More than 70% of patients with cancer develop significant pain at some time during the course of their illness. Many patients receive inadequate treatment for their pain. Most patients with advanced cancer often have multiple causes and sites of pain. Pain can be caused by direct tumor involvement of nerves (65-85%). Pain can also result from cancer therapy such as chemotherapy, surgery, or radiation treatment (15-25%). Between 3 and 10% of cancer patients can even have pain from non-cancer problems. Severe pain can be difficult to manage, particularly when a person is dying.

It is contemplated that the co-administration of tramadol and propoxyphene or the administration of a tramadol and propoxyphene co-formulation will be especially well suited to the treatment of cancer pain.

II. Pharmacokinetics

A. The Pharmacology of Tramadol

Tramadol is a synthetic 4-phenyl-piperidine analogue of codeine. It is a central acting analgesic with a low affinity for opioid receptors. Its selectivity for mu receptors has been demonstrated, and the M1 metabolite of tramadol, produced by liver O-demethylation, shows a higher affinity for opioid receptors than the parent drug. The rate of production of this M1 derivative (O-demethyl tramadol), is influenced by a polymorphic isoenzyme of the debrisoquine-type, cytochrome P450 2D6 (CYP2D6).

Nevertheless, this affinity for mu receptors of the CNS remains low, being 6000 times lower than that of morphine. Moreover, and in contrast to other opioids, the analgesic action of tramadol is only partially inhibited by the opioid antagonist naloxone, which suggests the existence of another mechanism of action. This was demonstrated by the discovery of a monoaminergic activity that inhibits noradrenaline (norepinephrine) and serotonin (5-hydroxytryptamine; 5-HT) reuptake, making a significant contribution to the analgesic action by blocking nociceptive impulses at the spinal level.
(+/−)-Tramadol is a racemic mixture of 2 enantiomers, each one displaying differing affinities for various receptors. (+/−)-Tramadol is a selective agonist of mu receptors and preferentially inhibits serotonin reuptake, whereas (−)-tramadol mainly inhibits noradrenaline reuptake. The action of these 2 enantiomers is both complementary and synergistic and results in the analgesic effect of (+/−)-tramadol.

After oral administration, tramadol demonstrates 68% bioavailability, with peak serum concentrations reached within 2 hours. The elimination kinetics can be described as 2-compartmental, with a half-life of 5.1 hours for tramadol and 9 hours for the M1 derivative after a single oral dose of 100 mg. This explains the approximately 2-fold accumulation of the parent drug and its M1 derivative that is observed during multiple dose treatment with tramadol. The recommended daily dose of tramadol is between 50 and 100 mg every 4 to 6 hours, with a maximum dose of 400 mg/day; the duration of the analgesic effect after a single oral dose of tramadol 100 mg is about 6 hours. Adverse effects, and nausea in particular, are dose-dependent and therefore considerably more likely to appear if the loading dose is high. The reduction of this dose during the first days of treatment is an important factor in improving tolerability. Other adverse effects are generally similar to those of opioids, although they are usually less severe, and can include respiratory depression, dysphoria and constipation.

Tramadol can be administered concomitantly with other analgesics, particularly those with peripheral action, while drugs that depress CNS function may enhance the sedative effect of tramadol. Tramadol should not be administered to patients receiving monoamine oxidase inhibitors, and administration with tricyclic antidepressant drugs should also be avoided. Tramadol has pharmacodynamic and pharmacokinetic properties that are highly unlikely to lead to dependence. This was confirmed by various controlled studies and post marketing surveillance studies, which reported an extremely small number of patients developing tolerance or instances of tramadol abuse. Tramadol is a central acting analgesic which has been shown to be effective and well tolerated, and likely to be of value for treating several pain conditions (step II of the World Health Organization ladder) where treatment with strong opioids is not required.

Selected embodiments of the present invention allow for administration of lower doses of tramadol, as a co-formulation with propoxyphene, while still providing adequate analgesia with a minimal side-effect profile.

B. The Pharmacology of Propoxyphene

Propoxyphene, is a synthetic, diphenylheptane-derivative opiate agonist which exerts a (centrally acting) analgesic effect. Propoxyphene shares structural similarities with methadone, the isomers of which have been suggested to have at least weak N-methyl-D-aspartate (NMDA) antagonist activity in vivo. A recent study suggests that in addition to activity at opiate receptors, propoxyphene also exhibits antagonist activity at the NMDA receptor. Propoxyphene is especially suited to the treatment of hyperalgesia of chronic pain associated with nerve or soft tissue injury and in the development of opioid tolerance both of which have been shown in animal experiments to be related to stimulation of the NMDA receptor.

Propoxyphene undergoes extensive dose-dependent first-pass metabolism and is quickly converted to an active metabolite, norpropoxyphene (NPP), which has very weak analgesic properties. Blood concentrations of NPP resemble those of DPP with a dramatic increase of the metabolite concentrations to the maximum at 2 to 4 hours.

Equimolar doses of propoxyphene hydrochloride or napsylate provide similar plasma concentrations. Peak plasma concentrations of propoxyphene are reached in 2 to 2.5 hours. After a 65-mg oral dose of propoxyphene hydrochloride, peak plasma levels of 0.05 to 0.1 mcg/ml are achieved.

Repeated doses of propoxyphene at 6-hour intervals lead to increasing plasma concentrations, with a plateau after the ninth dose at 48 hours. Propoxyphene is metabolized in the liver to yield norpropoxyphene. Propoxyphene has a half-life of 6 to 12 hours, whereas that of norpropoxyphene is 30 to 36 hours.

Selected embodiments of the present invention allow for administration of lower doses of propoxyphene, as a co-formulation with tramadol, while still providing adequate analgesia with a minimal side-effect profile.

III. Routes of Administration and Formulations

It is not intended that the present invention be limited by the particular nature of the therapeutic preparation. For example, a combined formulation comprising tramadol and propoxyphene can be provided together with physiologically tolerable liquid, gel or solid carriers, diluents, adjuvants and excipients. Formulations may also contain such normally employed additives as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions typically contain 1%-95% of active ingredient, preferably 2%-70%.

The present invention is not limited by the method of introduction of the therapeutic compound(s) to the body. Among other methods, the present invention contemplates administering the combined formulations comprising tramadol and propoxyphene topically, transdermally, orally, or by standard injection (e.g. sub-cutaneously or intravenously).

One embodiment of the present invention contemplates a sustained release formulation of tramadol and propoxyphene comprising a controlled release hydrophilic matrix system. This sustained release embodiment is not limited to any specific sustained release delivery system, and includes a formulation comprising a rapid release component, of propoxyphene and/or tramadol. The rapid release component may be incorporated into a dosage form in various ways. In one embodiment, said rapid release component comprises the outer coat of a tablet. In another embodiment, this rapid release component may be incorporated into a layer of a bi-layer tablet such that said bi-layer tablet has a rapid release layer and a controlled release layer.

These sustained release embodiments also include formulations comprising an immediate release component of propoxyphene and/or tramadol. Although it is not intended that the sustained release embodiment of the present invention be limited to a specific formulation or method of manufacture, examples of suitable sustained release formulations are described in U.S. Pat. No. 6,190,591 to Van Lengerich, et al. herein incorporated by reference.

Although the method by which the matrix system controls the release of pharmaceuticals in no way limits any embodiment of the present invention, it is believed that hydroxypropyl methylcellulose works by the outer surface of the tablet, caplet, etc. hydrating. Water permeates into the tablet, increasing the thickness of the outer layer causing it to become a gel and thereby limiting the escape of the pharmaceutical from the tablet.

In one specific embodiment, it is contemplated that the matrix system comprises hydroxypropyl methylcellulose. In another embodiment it is contemplated that the matrix system comprises Methocel™ (DOW, Edison, N.J.) hydroxypropyl methylcellulose. In a preferred embodiment, the matrix system comprises Methocel™ E, F or K hydroxypropyl methylcellulose. It will be noted that one practiced in the art may substitute other brands of hydroxypropyl methocellulose for Methocel™. The production of hydroxypropyl methocellulose based sustained-release and controlled-release pharmaceuticals is known to those practiced in the art (Using METHOCEL Cellulose Ethers for Controlled Release of Drugs in Hydrophilic Matrix Systems, DOW, Edison, N.J.). Additional examples of sustained-release formulations comprising hydroxypropyl methocellulose matrices are given in U.S. Pat. No. 4,389,393 to Schor, et al., which is incorporated herein by reference.

The present invention also contemplates administering combined formulations comprising tramadol and propoxyphene to the patient intranasally or through respiratory inhalation. Formulations suitable for intranasal administration include ointments, creams, lotions, pastes, gels, sprays, aerosols, oils and other pharmaceutical carriers which accomplish direct contact between tramadol and propoxyphene or a pharmaceutical composition comprising tramadol and propoxyphene and the nasal cavity. Examples of pharmaceutical compositions administered intranasally are described in U.S. Pat. Nos. 5,393,773 and 5,554,639 to Craig et al.; and U.S. Pat. No. 5,801,161 to Merkus, all hereby incorporated by reference. Formulations suitable for respiratory inhalation include, liquid or dry powder aerosols and other pharmaceutical carriers which accomplish direct contact between tramadol and propoxyphene or a pharmaceutical composition comprising tramadol and propoxyphene and the respiratory tract. Examples of pharmaceutical compositions administered through respiratory inhalation are described in U.S. Pat. No. 4,552,891 to Hu et al.; U.S. Pat. No. 5,869,479 to Kreutner et al., and U.S. Pat. No. 5,864,037 to Chasis et al., all hereby incorporated by reference. More specifically the devices described in U.S. Pat. Nos. 5,642,730, 5,964,223 and 6,079,413 are herein incorporated by reference for their teachings of an aerosolization means, capable of delivering an aerosol directly to the lung, which may be incorporated into selected embodiments of the present invention.

In some embodiments, intranasal administration and respiratory inhalation are the preferred modes of administration of the combined formulations due to the ease of administration and faster onset of therapeutic activity. It is contemplated that intranasal administration and respiratory inhalation are advantageous as they may allow a smaller effective dosage to be administered than would be possible with the oral route of administration. A preferred mode of administration comprises administration to the lung. Intrapulmonary delivery of pharmacologic agents to patients can be accomplished via aerosolization. Of course, the therapeutic agents may be investigated for their efficacy via other routes of administration, including parenteral administration.

Oral administration of tramadol and propoxyphene is a preferred route of administration. Peak plasma levels of tramadol are reached within 2 hours following oral administration and slightly longer for propoxyphene.

While the present invention is not limited by the form of oral administration, solid and liquid formulation of the tramadol and propoxyphene for oral administration are contemplated, one skilled in the art is able to readily prepare such solid formulations, and in one embodiment, the inactive ingredients include corn starch, croscarmellose sodium, hydroxypropyl methylcellulose, lactose, magnesium stearate, methocel ES, microcrystalline cellulose, povidine, propylene glycol, polyethylene glycol, polysorbate 80 and titanium dioxide.

Some embodiments contemplate rapidly dissolving oral dosage forms comprising tramadol and propoxyphene. In one preferred embodiment, the combined formulation comprising tramadol and propoxyphene will take the form of a rapidly dissolving dosage form. Rapidly dissolving dosage forms are described in Khankari et al., in U.S. Pat. No. 6,024,981, herein incorporated by reference. One skilled in the art would be able to adapt the dosage forms described Khankari et al. (supra) to comprise suitable dosages of tramadol and propoxyphene for oral administration to a subject in need thereof.

Briefly, the dosage form described in Khankari et al. (supra) is a hard, compressed, rapidly dissolvable dosage form adapted for direct oral dosing. The dosage form includes an active ingredient(s) and a matrix. The matrix is composed of at least a non-direct compression filler and a lubricant. The dosage form is adapted to rapidly dissolve in the mouth of a patient and thereby liberate the active ingredient. Preferably, the dosage form has a friability of about 2% or less when tested according to the U.S.P. The dosage form also preferably has a hardness of 15-50 Newtons (“N”).

It is desirable that the dosage form of Khankari et al. [supra] dissolve in about 60 seconds or less in the patient's mouth. It is also often desirable that the dosage form include at least one particle granule. The granule would be the active ingredient(s) and a protective material. In some cases, the protective material is a coating which serves to mask the taste of the active ingredient(s). These granules can include rapid release granules and or sustained release granules.

The active ingredient(s) are preferably in a particle, granular, microgranular or crystalline form protected by a protective material. This protective material can be an adsorbate, a microgranule such as disclosed in Sparks et al., U.S. Pat. No. 4,940,588, or a coating which forms microcapsules and/or microparticles as described in, without limitation, U.S. Pat. No. 5,178,878 to Wehling et al., herein incorporated by reference. Combinations of these are also contemplated, i.e., a coated adsorbate. In addition, protection can be provided by agglomeration or the formation of a matrix as is conventional. The dosage forms may also include a plurality of different active ingredients, each protected by a different means.

The protective materials described by Khankari et al. [supra] may include any of the polymers conventionally utilized in the formation of microparticles, matrix-type microparticles and microcapsules. Among these are cellulosic materials such as naturally occurring cellulose and synthetic cellulose derivatives; acrylic polymers and vinyl polymers. Other simple polymers include proteinaceous materials such as gelatin, polypeptides and natural and synthetic shellacs and waxes. Protective polymers may also include ethylcellulose, methylcellulose, carboxymethyl cellulose and acrylic resin material sold under the registered trademark EUDRAGIT by Rhone Pharma GmbH of Weiterstadt, Germany.

Rapid release dosage forms are those in which the drug is rapidly released from the encapsulant, coating or other protective material when desired. To the extent possible, the effect of the protective material under such circumstances will be minimal in terms of reducing the normal bioavailability of the same drug if unprotected. Thus, for example, where a coating is used to taste-mask the objectionable flavor of a material, it is important that that coating be intact, to the extent necessary to serve its taste-masking function, while the dosage form is in the mouth of the patient. However, once the patient has swallowed there is no longer a need to protect the tastebuds from the drug. It may be desirable that the drug be immediately bioavailable. In such a circumstance, it is desirable for the coating to either rupture in order to release its contents, dissolve thereby exposing its contents or allow the gastric juices in the stomach to permeate through and dissolve the active ingredient such that the bioavailability of the coated drug remains, as nearly as possible, the same as that of the same drug if administered in an unprotected form. Thus, if a tablet including nonprotected active ingredients would need normally to be dosed every four or every six hours, then the rapid release dosage form would also have to be administered on that same basis. A rapid release dosage form as described in Khankari et al. [supra] is one which disintegrates rapidly in the mouth to form a suspension of particles which, once they clear the mouth, will release their contents so as not to interfere with the normal bioavailability of the active ingredient(s).

The matrix of the dosage forms described in Khankari et al. [supra] includes at least two ingredients: a non-direct compression filler and a lubricant. The matrix will assist in preventing the rupture of any microcapsules, microparticles or other protected active ingredient incorporated therein during compression. The matrix will also assist in the rapid dissolution of the dosage form in the mouth. Finally, the matrix provides a positive organoleptic experience to the patient or subject.

Any conventional material can be used as a filler. The filler must be rapidly dissolvable when a tablet produced from the same is paced in the mouth. This means that the material must be significantly rapidly water soluble. In addition, generally, the particle size of the filler will be relatively small, particularly compared to direct compression fillers.

Particularly preferred fillers are non-direct compression sugars and sugar alcohols. Such sugars and sugar alcohols include, without limitation, dextrose, mannitol, sorbitol, lactose and sucrose. Of course, dextrose, for example, can exist as either a direct or compression sugar, i.e, a sugar which has been modified to increase its compressibility, or a non-direct compression sugar.

The amount of lubricant used can generally range from between about 1 to about 2.5% by weight. Hydrophobic lubricants useful in accordance with the present invention include alkaline stearates, stearic acid, mineral and vegetable oils, glyceryl behenate and sodium stearyl fumarate. Hydrophilic lubricants can also be used.

The method of manufacture of the orally disintegrable tablet as described in Khankari et al. [supra] includes the steps of forming a mixture of the active ingredient(s) and the matrix; and compressing the mixture to form a plurality of hard, compressed, rapidly disintegrable tablets adapted for direct oral dosing. Preferably, tablets are formed by “direct compression” (i.e. such that one can avoid the difficulty and expense of a wet or dry granulation prior to compression). The tablets will preferably have a hardness of at least about 15 Newtons and will be adapted to dissolve in the mouth of a patient within about 90 seconds to liberate the particles. In most formulations, the hardness may be at least 20 Newtons and the tablet dissolves in 45 seconds or less. Other conventional tableting or slugging methods known in the art are also contemplated. Indeed, any method in which a mixture of the active ingredient, often in the form of a protected particle, and the matrix are compressed into a solid dosage form having the properties disclosed herein are acceptable. After tableting or slugging, the dosage forms can be packaged in the lumen of a package or stored in bulk.

In addition to the ingredients previously discussed, the matrix as described in Khankari et al. [supra] may also include wicking agents, non-effervescent disintegrants and effervescent disintegrants. Wicking agents are compositions which are capable of drawing water up into the dosage form. They help transport moisture into the interior of the dosage form. In that way the dosage form can dissolve from the inside, as well as from the outside.

In general, the total amount of wicking agents, non-effervescent disintegrants and effervescent disintegrants should range from 0-50%. However, it should be emphasized that the formulations will dissolve rapidly and therefore, the need for disintegration agents is minimal.

A non-limiting representative oral rapidly dissolving tablet comprising tramadol and propoxyphene can be prepared by one skilled in the art as described in Example 4 of Khankari et al. [supra], by removing the coated paracetamol and using tramadol and propoxyphene in appropriate amounts as the active ingredients. The tramadol and propoxyphene can be suitably coated as described in Khankari et al. [supra]. The resulting tablets are expected to rapidly dissolve with a minimum of grit and a pleasant organoleptic experience.

In another embodiment, the active ingredients (i.e. tramadol and propoxyphene) will be incorporated into granulates for incorporation into a fast-disintegrating and dissolving compositions. Granulates and fast-disintegrating and dissolving compositions are described in U.S. Pat. No. 5,837,292 to Dijkgraaf et al., herein incorporated by reference. Briefly, by making a blend of a substantially water soluble drug and at least 15 weight percent by volume (in water), the percentage based on the weight of the drug, granulating the same with water and mixing the granules so obtained after drying with suitable excipients, such as disintegrants, lubricants, flavors and sweetening agents, in an amount as low as possible, fast-disintegrating and dissolving compositions can be prepared. More particularly such compositions, preferably containing a high amount of drug as well, comprise the above described granulate in an amount of at least 80 wt % in admixture with 2-8 wt % of a second disintegrant, the percentages based on the weight of the composition. Optionally the compositions may also contain flavors, sweetening agents, such as saccharinc acid, the sodium salt thereof or aspartame, lubricants, such as colloidal silicon dioxide, stearic acid or a salt thereof, etc.

The water dispersible hydrocolloid to be used in the granulates and compositions as described in Dijkgraaf et al. [supra] may be from an inorganic source, such as expanding lattice clays, like bentonite or montmorillonite. It can also be an organic substance such as a water dispersible cellulose, also known as microcrystalline cellulose and carboxymethyl cellulose sodium in the U.S. Pharmacopoeia/National Formulary. Four types of water dispersible celluloses, which are colloidal forms of microcrystalline cellulose, prepared by chemical depolymerization of highly purified wood pulp, the original crystalline areas of the fibers being combined with sodium carboxymethyl cellulose and spray-dried, have been marketed under the trade names Avicel® RC-501 (containing 7.1-11.9% of sodium carboxylmethyl cellulose), Avicel® RC-581 (containing 8.3-13.8% of sodium carboxymethyl cellulose), Avicel® RC-591 (containing 8.3-13.8% of sodium carboxymethyl cellulose) and Avicel® CL-611 (containing 11.3-18.8% of sodium carboxymethyl cellulose). All types are hygroscopic powders, which are insoluble in organic solvents and dilute acids, and partially soluble in both dilute alkali and water (due to the sodium carboxymethyl cellulose component). Although all four types may be used to prepare the granulate according to the method of Dijkgraaf et al. [supra], preferably the Avicel® RC-581 type is used, but most advantageously the Avicel® RC-591 type is incorporated in the granulate in an amount of approximately 15 wt %. Preferably the hydrocolloid is used in a concentration of between 1 and 10 wt %, but more advantageously in a concentration ranging from 2 to 5 wt %, all percentages based on the weight of the drug.

The granulates according to the Dijkgraaf et al. [supra] are prepared according to methods known in the art. Preferably the drug and water dispersible hydrocolloid are blended and water is added until the material is sufficiently wetted. The amount of water used may range from 20 to 30 wt %, based on the weight of the granulate. After partial drying the wet mass is passed through a first screen and subsequently further dried in a fluidized bed dryer at an air inlet temperature of between 40° C. and 60° C. After drying the granules are passed through a second screen. Alternatively the wet mass is transferred to a fluidized bed dryer without wet screening. After drying the granules are passed through a first and a second screen and optionally a third screen respectively.

The compositions, based on the above granulate of Dijkgraaf et al. [supra], preferably contain the granulate in an amount of approximately 80 wt % in order to comply with the requirement to provide a high-dosed composition. It goes without saying that the granulate can also be incorporated in a dosage-form together with a greater part of excipients.

In order to prepare fast-disintegrating and fast-dissolving compositions as described in Dijkgraaf et al. [supra], containing a high amount of drug, the granulate is advantageously blended with a first disintegrant and a second disintegrant and optionally other excipients such as a lubricant, flavors and sweetening agents. The first disintegrant is preferably a cellulose product, which is microcrystalline cellulose (Avicel® PH 101, Avicel® PH 102), microfine cellulose or a mixture thereof. The second disintegrant is selected from the group of superdisintegrants, such as cross-linked polyvinylpyrrolidone and low-substituted hydroxypropyl cellulose. Both the first and the second disintegrant are advantageously added in an amount of 2-8 wt %, more preferably 3-6 wt %, the percentage based in the weight of the composition. Most preferably the ratio of the amount of the first and the second disintegrant in the composition is 1:1.

By fast disintegration of the compositions according to Dijkgraaf et al. [supra] is meant a disintegration time in water of room temperature of less than 2 minutes and preferably less than one minute. Fast dissolution is to be considered as >95% of the drug dissolved in water of 37° C. after 30 minutes. Preferably 90% of the drug has been dissolved after 10 minutes (same conditions).

Further, tramadol and propoxyphene may be formulated in tablets, lozenges or pills suitable for administration to the oral mucosa (e.g. buccal or sublingual delivery formulations). Such mucosal delivery forms permit absorption of tramadol and propoxyphene directly through the buccal or sublingual mucosa of the oral cavity. In certain instances, such absorption may be desirable.

In one embodiment, the dosage form is particularly suited for buccal administration, as described in U.S. Pat. No. 5,244,668 to Snipes, herein incorporated by reference. Snipes [supra] provides an excipient for a pharmaceutical compound which melts at body temperature but will not spontaneously deform at higher temperatures encountered in shipment and storage. The excipient of Snipes [supra] comprises: (i) low molecular weight polyethylene glycol (melting point about 37° C.) (75-90% of the excipient); (ii) medium to high molecular weight polyethylene glycol (0-4% of the excipient); (iii) a long chain saturated carboxylic acid (0-4% of the excipient); (iv) polyethylene oxide (molecular weight 100,000-500,000) (0-4% of the excipient) and (v) colloidal silica (10-20% of the excipient) (wherein all percentages are by weight). The excipient of Snipes [supra] is a water soluble matrix composition for containing a pharmaceutically active ingredient and which softens essentially to an easily flowable material at body temperature, yet can be molded into unit dosage forms which maintain their shape under the temperature extremes and handling which occur in the normal course of commercial distribution and sale of the medications. One of skill in the art would be able to produce a buccally rapidly dissolving dosage form comprising tramadol and propoxyphene by adapting the teaching provided by Snipes [supra].

Some of these transmucosal dosage forms comprise rapidly dissolving tablets, lozenges or pills. In one embodiment, the combined formulation comprising tramadol and propoxyphene is in a fast dissolving buccal dosage form. A fast-dissolving dosage form is described in U.S. Pat. No. 5,122,616 and U.S. Pat. No. 5,073,374 to McCarty, both herein incorporated by reference. The fast dissolving buccal formulation may include essentially three components: the active ingredients (i.e. tramadol and propoxyphene for the purposes of the present invention), a pharmaceutically acceptable lubricant and a soluble, directly compressible tablet excipient.

The soluble excipient as described in McCarty [supra] is typically a sugar, such as sucrose or lactose. The preferred sugar is sorbitol, and in particular, sorbitol N.F. and/or spray dried sorbitol in an amount ranging from about 90 to 99 percent. The soluble excipients also include vehicles for hydrophobic actives. Such vehicles include solids which melt at about room temperature and surfactants. Suitable surfactants include Pluronic, Tweens, sodium lauryl sulfate, and the like, and suitable liquefying solids include the various polyethylene glycols, low melting glycerides, and various suppository bases, which are known to one skilled in the art. The lubricant used in the fast buccal formulation may be any conventional lubricant, such as magnesium stearate or sodium dodecyl sulfate. Generally, the lubricant should be water soluble. Hence, the preferred lubricant is sodium dodecyl sulfate in an amount ranging from about 1 to 3 percent.

The rapidly dissolving buccal formulations of McCarty [supra] can be prepared by simply mixing the ingredients together and compressing desired amounts of the mixture into tablet form. The final formulations desirably have a diameter of about 0.635 cm and a thickness of about 0.127 cm, and upon administration disintegrate in about 30 seconds to around 5 minutes, and preferably in about one minute.

In one embodiment, a rapidly dissolving dosage form, suitable for oral administration is contemplated. Such a dosage form, which incorporates an effervescent disintegration agent and microparticles, is described in U.S. Pat. No. 5,178,878 to Wehling et al., herein incorporated by reference. Briefly, Wehling et al. [supra] provide a solid pharmaceutical dosage form which includes a mixture incorporating at least one water and/or saliva activated effervescent disintegration agent and microparticles. The microparticles incorporate a pharmaceutical ingredient together with a protective material substantially encompassing the pharmaceutical ingredient. The protective material substantially shields the pharmaceutical ingredient from contact with the environment outside of the microparticle.

The microparticles in each dosage form desirably contain an effective amount of at least one systematically distributable pharmaceutical ingredient. The mixture including the microparticles and effervescent agent desirably is present as a tablet of a size and shape adapted for direct oral administration to a patient, such as a human patient. The tablet is substantially completely disintegrable upon exposure to water and/or saliva. The effervescent disintegration agent is present in an amount effective to aid in disintegration of the tablet, and to provide a distinct sensation of effervescence when the tablet is placed in the mouth of a patient.

The effervescent sensation is not only pleasant to the patient but also tends to stimulate saliva production, thereby providing additional water to aid in further effervescent action. Thus, once the tablet is placed in the patient's mouth, it will disintegrate rapidly and substantially completely without any voluntary action by the patient. Even if the patient does not chew the tablet, disintegration will proceed rapidly. Upon disintegration of the tablet, the microparticles are released and can be swallowed as a slurry or suspension of the microparticles. The microparticles thus may be transferred to the patient's stomach for dissolution in the digestive tract and systemic distribution of the pharmaceutical ingredient(s).

One embodiment contemplates an orally administrable dosage form in which the active ingredient(s) are in combination with an effervescent agent used as a penetration enhancer to influence the permeability of the active ingredient(s) across the buccal, sublingual, and gingival mucosa. Such a dosage form is described by Pather et al. [U.S. Pat. No. 6,200,614 B1], herein incorporated by reference.

Briefly, the dosage forms described by Pather et al. [supra] should include an amount of an effervescent agent effective to aid in penetration of the drug across the oral mucosa. Preferably, the effervescent is provided in an amount of between about 5% and about 95% by weight, based on the weight of the finished tablet, and more preferably in an amount of between about 30% and about 80% by weight.

The term “effervescent agent” includes compounds which evolve gas. The preferred effervescent agents of Pather et al. [supra] evolve gas by means of a chemical reaction which takes place upon exposure of the effervescent agent (an effervescent couple) to water and/or to saliva in the mouth. This reaction is most often the result of the reaction of a soluble acid source and a source of carbon dioxide such as an alkaline carbonate or bicarbonate. The acid sources may be any which are safe for human consumption and may generally include food acids, acid and hydrite antacids such as, for example: citric, tartaric, amalic, fumeric, adipic, and succinics. Carbonate sources include dry solid carbonate and bicarbonate salt such as, preferably, sodium bicarbonate, sodium carbonate, potassium bicarbonate and potassium carbonate, magnesium carbonate and the like. Reactants which evolve oxygen or other gasses and which are safe for human consumption are also included.

In addition to the effervescence-producing agents, a dosage form according to Pather et al. [supra] may also include suitable non-effervescent disintegration agents. Non-limiting examples of non-effervescent disintegration agents include: microcrystalline, cellulose, croscarmelose sodium, crospovidone, starches, corn starch, potato starch and modified starches thereof, sweeteners, clays, such as bentonite, alginates, gums such as agar, guar, locust bean, karaya, pecitin and tragacanth. Disintegrants may comprise up to about 20 weight percent and preferably between about 2 and about 10% of the total weight of the composition.

The dosage forms of Pather et al. [supra] may also include glidants, lubricants, binders, sweeteners, flavoring and coloring components. Any conventional sweetener or flavoring component may be used. Combinations of sweeteners, flavoring components, or sweeteners and flavoring components may likewise be used.

Examples of binders which can be used in the dosage forms of Pather et al. [supra] include acacia, tragacanth, gelatin, starch, cellulose materials such as methyl cellulose and sodium carboxy methyl cellulose, alginic acids and salts thereof, magnesium aluminum silicate, polyethylene glycol, guar gum, polysaccharide acids, bentonites, sugars, invert sugars and the like. Binders may be used in an amount of up to 60 weight percent and preferably about 10 to about 40 weight percent of the total composition.

Coloring agents according to Pather et al. [supra] may include titanium dioxide, and dyes suitable for food such as those known as F.D.& C. dyes and natural coloring agents such as grape skin extract, beet red powder, beta-carotene, annato, carmine, turmeric, paprika, etc. The amount of coloring used may range from about 0.1 to about 3.5 weight percent of the total composition.

Flavors incorporated in the composition as described by Pather et al. [supra] may be chosen from synthetic flavor oils and flavoring aromatics and/or natural oils, extracts from plants, leaves, flowers, fruits and so forth and combinations thereof. These may include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Also useful as flavors are vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. The amount of flavoring may depend on a number of factors, including the organoleptic effect desired. Flavors may be present in an amount ranging from about 0.05 to about 3 percent by weight based upon the weight of the composition. Particularly preferred flavors are the grape and cherry flavors and citrus flavors such as orange.

One aspect of the dosage form according to Pather et al. [supra] provides a solid, oral tablet dosage form suitable for sublingual, buccal, and gingival administration. Excipient fillers can be used to facilitate tableting. The filler desirably will also assist in the rapid dissolution of the dosage form in the mouth. Non-limiting examples of suitable fillers include: mannitol, dextrose, lactose, sucrose, and calcium carbonate. One of skill in the art would be able to prepare an orally administratable dosage form comprising tramadol and propoxyphene with enhanced permeability of tramadol and propoxyphene across the buccal, sublingual and gingival mucosa by adapting the teaching provided by Pather et al. [supra].

In another embodiment, the active ingredients (i.e. tramadol and propoxyphene) are prepared in a rapidly dissolving dosage form, suitable for buccal administration. Rapidly dissolving dosage forms suitable for buccal administration are described in U.S. Pat. No. 5,576,014 to Mizumoto et al., herein incorporated by reference. These dosage forms take the form of a compressed molding comprising a saccharide having low moldability and saccharide having a high moldability, and show quick dissolution and disintegration in the buccal cavity, while having an adequate hardness for handling.

Examples of low moldability saccharides include lactose, mannitol, glucose, sucrose, xylitol, and the like, of which lactose and mannitol are preferred. These saccharides may be used alone, or as a mixture of two or more. Examples of high moldability saccharides include maltose, maltitol, sorbitol, oligosaccharides and the like, of which maltose and maltitol are preferred. These saccharides may be used alone or as a mixture of two or more.

The buccally dissolving compressed molding described by Mizumoto et al. [supra] uses a saccharide having low moldability as its main component, with a blending ratio of a high moldability saccharide to the low moldability saccharide being from 2 to 20% by weight, preferably from 5 to 10% by weight. Preferably, granules obtained by granulating lactose and/or mannitol which has low moldability with maltose or maltitol which has high moldability in an amount of from 5 to 7.5% by weight based on the total weight of the buccally dissolving compressed molding are used for the preparation of the molding as described by Mizumoto et al. [supra].

The active ingredient(s) may be mixed by a number of ways, as described in Mizumoto et al. [supra]. Preferably, the active ingredient may be used in an amount of 50% (w/w) or less, preferably 20% (w/w) or less, based on the total solid components (i.e. the total amount of the preparation), though it varies depending on the nature of each active ingredient to be used. The compressed moldings described by Mizumoto et al. [supra] may contain various additive agents generally used in the production of tablets, as long as they do not spoil the effects of the present invention. Such additive agents include disintegrating agents, binding agents, scouring agents, vesicants, artificial sweeteners, perfumes, lubricants, coloring agents, and the like, as described in Mizumoto et al. [supra].

The buccally compressed moldings of Mizumoto et al. [supra] are produced through conventionally used production steps, namely granulation and tableting, without employing a freeze drying step. One of skill in the art would be able to use and adapt the compressed moldings of Mizumoto et al. [supra] to prepare a rapidly intrabuccally dissolving compressed molding comprising tramadol and propoxyphene.

Combined formulations of propoxyphene and tramadol may also be administered transdermally in a carrier adapted for topical administration. Such carriers include creams, ointments, lotions, pastes, jellies, sprays, aerosols, bath oils, or other pharmaceutical carriers which accomplish direct contact between tramadol and propoxyphene and the pore of the skin. In general, pharmaceutical preparations may comprise from about 0.001% to about 10%, and preferably from about 0.01 to 5% by w/w of the active compound(s) (e.g., tramadol and propoxyphene) in a suitable carrier. In some cases it may be necessary to dissolve the active compound(s) in an appropriate solvent such as ethanol or DMSO (dimethylsulfoxide), and the like, to facilitate incorporation into a pharmaceutical preparation. Such preparations may also be incorporated into a “patch” for topical transcutaneous and transdermal delivery.

While the present invention is not limited by a specific method of introducing combined formulations comprising tramadol and propoxyphene by injection, injection of tramadol and propoxyphene can be carried out by any conventional injection means (e.g., employing an hypodermic syringe and needle or a similar device such as the NovolinPen. sold by Squibb-Novo, Inc., Princeton, N.J., USA). This injection may be by the subject injecting him or herself or by another person injecting the subject.

Propoxyphene and tramadol can be introduced by injection in a physiologically acceptable composition. Such compositions are aqueous solutions that are physiologically acceptable for administration by injection. The physiologically acceptable carrier is selected such that it is not painful or irritating upon injection. The physiologically acceptable compositions will preferably be sterile at the time of administration by injection.

Among the physiologically acceptable compositions for use in the methods is physiological saline or phosphate buffered saline, in which tramadol and propoxyphene are dissolved or suspended, such that the resulting composition is suitable for injection. Such a physiologically acceptable composition can also include a non-irritant preservative, such as, e.g., benzalkonium chloride at 0.05% (w/v) to 0.2% (w/v).

While the present invention is not limited to the method of injecting tramadol and propoxyphene, in the preferred embodiment, they are injected with a standard syringe or by an intravenous drip. One skilled in the art would be capable of injecting tramadol and propoxyphene with a carrier as described above.

IV. Dosages

While the present invention is not limited to a specific dosage level, for adult humans, the dosages of the tramadol and propoxyphene are appropriately between one percent and 80 percent of the normally prescribed dose of each when used in monotherapy for the treatment of analgesia, more preferably between ten percent and seventy five percent of the normally prescribed dose of each when used in monotherapy for the treatment of pain. In monotherapy for the treatment of pain, tramadol is generally prescribed at a dose of 50 mg or a dose of 100 mg every 4 to 6 hours (not to exceed 400 mg/day), while propoxyphene is generally prescribed at a dose of 65 mg every 4 hours as needed for pain (usually, not to exceed 395 mg/day). Of course, other schemes are possible and the invention is not limited to these specific dosages.

As demonstrated in the “Examples” section below certain combinations of tramadol and propoxyphene result in hyperalgesia. See, for example Group 8 in Table 3 (i.e. ED₂₅ tramadol ED₂₅ propoxyphene). That is to say, there is an increased sensitivity to pain) when a co-formulation of tramadol and propoxyphene is administered at these lower dosages (e.g. ED₂₅ tramadol ED₂₅ propoxyphene and ED₁₅ tramadol ED₃₅ propoxyphene). This hyperalgesia, however, was replaced by a supra-analgesia at propoxyphene ED doses greater than ED₄₀. In one embodiment of the present invention, a formulation corresponding to 1.24 mg/kg tramadol and 7.9 mg/kg propoxyphene in mice (corresponding to an ED₁₀ tramadol ED₄₀ propoxyphene) resulted in a significant 69.4% analgesia as compared to the expected analgesia of 50.0%. An inverse tramadol/propoxyphene dosage of 3.13 mg/kg tramadol and 3.09 mg/kg propoxyphene (corresponding to a ED₄₀ tramadol ED₁₀ propoxyphene) resulted in a 58% analgesia over the expected 50%.

V. Treatment Regimens

For the treatment of pain, it is envisioned that the prescribing physician will initiate treatment with a low dosage combined formulation comprising tramadol and propoxyphene. For example, a subject may initiate treatment with a formulation such as sample formulation S. If the symptoms of pain do not improve within three consecutive administrations, the subject may then try another formulation (for example formulation N, if the chief complaint is breakthrough pain. Further alterations in the dosage of the formulation may be made as necessary.

### EXAMPLES

The following examples serve to illustrate certain preferred embodiments and aspects of the present invention and are not to be construed as limiting the scope thereof.

In the disclosure which follows, the following abbreviations apply: eq (equivalents); M (Molar); 1M (micromolar); N (Normal); mol (moles); mmol (millimoles); μmol (micromoles); nmol (nanomoles); g (grams); mg (milligrams); μg (micrograms); L (liters); ml (milliliters); μl (microliters); cm (centimeters); mm (millimeters); μm (micrometers); nm (nanometers); ° C. (degrees Centigrade).

### I. EXEMPLAR FORMULATIONS

Examples 1-3 are directed towards preferred formulations of a tramadol/propoxyphene co-formulation. These formulations are not intended to limit the ways in which tramadol/propoxyphene may be alternatively formulated according to the teachings set out in the instant application.

### Example 1

This example describes the preparation of rapidly dissolving buccal tablets comprising tramadol and propoxyphene. See, U.S. Pat. No. 5,122,616 and U.S. Pat. No. 5,073,374 herein incorporated by reference.

The following ingredients are blended using a V-blender with an intensifier bar and are mixed for about five to ten minutes.

| Ingredient | Amount mg/tablet (% by Weight) |
|---|---|
| Propoxyphene | 60.0 |
| Tramadol | 5.0 |
| Sorbitol N.F. | 445.0 |
| Sodium Dodecyl Sulfate | 1.0 |

Tablets weighing about 100 mg/tablet are formed using a compression force of about 1000 PSI.

### Example 2

This example describes an effervescent dosage form with microparticles comprising tramadol and propoxyphene. See, Wehling et al. [supra] 374 herein incorporated by reference. The following ingredients are employed to form microparticles:

| Ingredient | Weight (g) | % Weight |
|---|---|---|
| EUDRAGIT RL-30-D | 267.5 | 26.8 |
| Propoxyphene | 60.0 | 6.0 |
| Tramadol | 5.0 | 0.5 |
| Mannitol | 637.5 | 63.8 |
| Magnesium Oxide | 30.0 | 3.0 |

The EUDRAGIT material is furnished by the manufacturer as a dispersion containing 30% solids (polymer) in water. The quantity needed to provide 267.5 grams is placed in a beaker and mixed to form a vortex. The mannitol, propoxyphene and tramadol are added and mixing is continued for 10 minutes. After this 10 minute mixing period, the magnesium oxide is added and mixing is continued another 10 minutes. These mixing steps are to take place at room temperature. The resulting mixture is poured into a tray and dried in an oven at 50° C. under air for one hour. After one hour, the resulting partially dried mixture is broken into lumps and then dried for an additional hour at 50° C. The dried lumps are then comminuted to microparticles, and screened through an 8 mesh screen. The screened microparticles are dried for an additional hour at 60° C.

The fraction of the resulting microparticles passing through a 30 mesh screen is collected. A portion of the microparticles are tableted into an effervescent tablet of about 1.0-2.0 kilo pounds hardness with an effervescent disintegration agent and other ingredients according to the following recipe:

| Ingredient | mg/tablet |
|---|---|
| Mannitol | 225.0 |
| Aspartame | 40.0 |
| Cherry Flavor | 6.0 |
| Magnesium stearate | 5.0 |
| Silicon dioxide | 1.0 |
| Sodium bicarbonate | 100.0 |
| Citric acid | 80.0 |
| Microparticles | 94.3 |

The effervescent tablets should have a dissolution time of less than about one minute. When administered by mouth, the effervescent tablets will provide prompt bioavailability of the tramadol and propoxyphene.

### Example 3

This example provides a rapidly dissolving dosage form comprising tramadol and propoxyphene particularly suitable for buccal administration. See, U.S. Pat. No. 5,244,668 to Snipes 374 herein incorporated by reference.

Buccal tablets are prepared by melting a suitable amount of polyethylene glycol (molecular weight 1000) and maintaining it at approximately 75° C. To this are added appropriate amounts of polyethylene glycol (molecular weight 8000) and myristic acid. The mixture is stirred for approximately 5 minutes. An appropriate amount of polyethylene oxide (molecular weight 5,000,000) is then slowly added and stirred for approximately 45 minutes to effect dissolution. Next, the appropriate amount of colloidal silica is added, and the desired amounts of tramadol and propoxyphene. The mixture is blended until smooth and homogenous. The matrix is then spread in a thin layer and allowed to solidify at room temperature. A portion of the hardened matrix is granulated and fed into an injection molding machine. By this process, buccal tablets comprising tramadol and propoxyphene and having a thin elongated oval shape can be prepared. Each injection molded buccal tablet weighs approximately 79.50 mg and, in one embodiment, it is contemplated that a preferred dosage of said buccal tablet comprises the administration of two tablets with the following composition:

| Ingredient | Amount [mg (approximate % by weight)] |
|---|---|
| Polyethylene glycol | 38.25 (48.05) |
| (Molecular Weight 1000) | |
| Polyethylene glycol | 0.25 (0.314) |
| (Molecular Weight 8000) | |
| Myristic acid | 0.25 (0.314) |
| Polyethylene oxide | 0.25 (0.314) |
| Tramadol | 2.50 (3.14) |
| Propoxyphene | 30.00 (37.74) |
| Colloidal silica | 8.00 (10.06) |

II. In Vivo Data

Examples 4-7 describe protocols and present data evaluating the dose-additivity of the analgesic effects of tramadol and dextro-propoxyphene, in a validated animal model.

### Example 4

### Preparation Of Compounds

In the animal studies presented in Examples 5-7, tramadol and dextro-propoxyphene were administered neat (undiluted), or were diluted with diluted with saline (i.e. 0.9% Sodium Chloride for injection) in order to achieve the desired dose volumes. Fresh solutions were prepared for each concentration (of each compound) prior to administration.

Acetic acid was used to assess experimental pain or “writhing.” Specifically, thirty minutes after dosing, the writhing-induction agent (0.6% Acetic Acid) was administered to all experimental groups by intraperitoneal injection at a dose volume of 10 mL/kg.

### Example 5

### Animals

Crl:CFW® (SW) BR mice, from Charles River Laboratories were used as the animal model to test the analgesia of the tramadol/propoxyphene co-formulations. The test animals were approximately 4 weeks of age on arrival and each animals weighed approximately 22 to 28 g within 3 days of arrival. Each animal used had a body weight within ±20% of the mean body weight for each sex. Animals considered suitable for study were weighed prior to treatment and randomized, by sex, into treatment groups using a standard, by weight, block randomization procedure.

All animals were permitted an acclimation period of approximately 1 week. During this acclimation period, all animals were observed daily for any clinical signs of disease and all animals will be given a detailed clinical examination prior to selection for study. All animals with any evidence of disease or physical abnormalities were euthanized using carbon dioxide inhalation and discarded.

Upon receipt the animals were housed 3-4 per cage for several days to allow time to adapt to the automatic watering system. Subsequently, the animals will be caged individually in suspended, stainless steel, wire-mesh type cage.

Fluorescent lighting was provided via an automatic timer for approximately 12 hours per day. Temperature and humidity will be monitored and recorded daily and maintained to the maximum extent possible between 64 to 79° F. and 30 to 70%, respectively.

The basal diet was block Lab Diet® Certified Rodent Diet #5002, PMI Nutrition International, Inc. This diet was be available ad libitum. Tap water was supplied ad libitum via an automatic water system.

### Example 6

### Study Design

The protocols employed assessed the qualitative and quantitative changes in abdominal muscle contractions (writhing) induced after the subcutaneous administration of a noxious chemical stimulus, 0.6% acetic acid, in mice. The degree and amount of contractile abdominal activity is a measure of “experimentally-induced pain”. A decrease in the amount of writhing with prior administration of a test article is an experimental measure of “analgesia.”

Dose-response functions were generated for each of the two (2) test articles (tramadol, dextro-propoxyphene,). ED₅₀ values for analgesic activity were estimated using a least squares linear regression analysis procedure using PHARM/PCS or SAS.

### Example 7

### Dosage and Observation/Dose-Response

The test article, positive control, and vehicle were administered to all groups as a single subcutaneous injection in the scapular region on the back at a dose volume of 10 mL/kg and at the dose levels as presented in Table 1 below.

TABLE 1
Group Assignment

| Group | | Number of |
|---|---|---|
| Number | Dose Level | Male Animals |
| 1 | 0 mg/kg | 10 |
| 2 | 0.32 mg/kg Tramadol | 10 |
| 3 | 1 mg/kg Tramadol | 10 |
| 4 | 3.2 mg/kg Tramadol | 10 |
| 5 | 1 mg/kg d-Propoxyphene | 10 |
| 6 | 3.2 mg/kg d-Propoxyphene | 10 |
| 7 | 10 mg/kg d-Propoxyphene | 10 |
| 8 | 2.19 mg/kg Tramadol + 5.49 mg/kg | 10 |
| | d-Propoxyphene | |
| 9 | 1.56 mg/kg Tramadol + 7.09 mg/kg | 10 |
| | d-Propoxyphene | |
| 10 | 1.24 mg/kg Tramadol + 7.90 mg/kg | 10 |
| | d-Propoxyphene | |
| 11 | 1.24 mg/kg Tramadol + 7.90 mg/kg | 10 |
| | d-Propoxyphene | |
| 12 | 0.93 mg/kg Tramadol + 8.73 mg/kg | 10 |
| | d-Propoxyphene | |
| 13 | 3.13 mg/kg Tramadol + 3.09 mg/kg | 10 |
| | d-Propoxyphene | |

Thirty minutes after dosing, the writhing-induction article, 0.6% acetic acid, was administered to all groups by intraperitoneal injection at a dose volume of 10 mL/kg.

Immediately after administration of the writhing-induction article, animals were placed into a poly-observation chamber and a 15-minute evaluation period was initiated. During the 15-minute evaluation period, the number of “writhes” that occurred were recorded. For scoring purposes, a “writhe” was indicated by whole body stretching or contraction of the abdomen, further described as waves of contraction of the abdominal musculature, twisting and turning of the trunk, and hind-limb extension. The alleviation of “pain” or analgesia was determined by the dosage at which 50% of the mice in a test group exhibited an analgesic response for the article or article combination be tested.

All animals were observed at least twice a day for morbidity, mortality, injury, and availability of food and water. Any animals in poor health were identified for further monitoring and possible euthanasia. Any animal showing signs of severe debility or toxicity, particularly if death appears imminent, was be euthanized for humane reasons using the anesthesia method as described under Euthanasia in the Test System portion of this protocol. Body weights were measured and recorded within 3 days of arrival, at least once prior to randomization, and prior to dosing.

Table 2 presents the dose-response from the administration of vehicle, tramadol, or propoxyphene.

TABLE 2

| Drug | Dose | | |
|---|---|---|---|
| Treatment | (mg/kg) | Ave. # of writhes | % Analgesia |
| Saline | 0 | 52.9 | 0 |
| Tramadol | 0.32 | 54.6 | −3.21 |
| Tramadol | 1.0 | 55.1 | −4.16 |
| Tramadol | 3.2 | 31.7 | 40.08 |
| Propoxyphene | 1.0 | 59.4 | −12.29 |
| Propoxyphene | 3.2 | 45.4 | 14.18 |
| Propoxyphene | 10.0 | 26.7 | 49.53 |

Table 3 presents drug interaction data for various combinations of tramadol and propoxyphene on analgesia.

TABLE 3

| | Tramadol | Propoxyphene | | |
|---|---|---|---|---|
| | Dose | Dose | Ave. # | % |
| | (mg/kg) | (mg/kg) | of writhes | Analgesia |
| Group 8 | | | | |
| ED_{25Tramadol} + | 2.19 | 5.49 | 37.0 | 30.06 |
| ED_{25Propoxyphene} | | | | |
| Group 9 | | | | |
| ED_{15Tramadol} + | 1.56 | 7.09 | 36.3 | 31.38 |
| ED_{35Propoxyphene} | | | | |
| Group 10 | | | | |
| ED_{10Tramadol} + | 1.24 | 7.9 | 18.4 | 65.22 |
| ED_{40Propoxyphene} | | | | |

Table 4 presents a second series of interaction tests for various combinations of tramadol and propoxyphene on analgesia.

TABLE 4

| | Tramadol | Propoxyphene | | |
|---|---|---|---|---|
| | Dose | Dose | Ave. # | % |
| | (mg/kg) | (mg/kg) | of writhes | Analgesia |
| Group 11 | | | | |
| ED_{10Tramadol} + | 1.24 | 7.90 | 16.2 | 69.37 |
| ED_{40Propoxyphene} | | | | |
| Group 12 | | | | |
| ED_{5 Tramadol} + | 0.93 | 8.73 | 17.5 | 66.91 |
| ED_{45Propoxyphene} | | | | |
| Group 13 | | | | |
| ED_{40 Tramadol} + | 3.13 | 3.09 | 22.1 | 58.2 |
| ED_{10Propoxyphene} | | | | |

1. A method for the treatment of pain comprising:
a) providing:
i) a subject with pain;
ii) a combined formulation comprising tramadol and propoxyphene; and,
b) administering said combined formulation to said subject such that said pain is reduced.

2. The method of claim 1, wherein said subject has been previously treated with tramadol monotherapy.

3. The method of claim 1, wherein said combined formulation of tramadol and propoxyphene is formulated as a tablet suitable for oral administration.

4. The method of claim 1, wherein said combined formulation of tramadol and propoxyphene is formulated as a tablet further comprising a controlled release solid dosage form suitable for oral administration.

5. The method of claim 4, wherein said controlled release solid dosage form further comprises an initial rapid release component.

6. The method of claim 1, wherein said combined formulation of tramadol and propoxyphene is formulated as tablet further comprising a rapidly dissolving solid dosage form suitable for oral administration.

7. The method of claim 1, wherein said combined formulation of tramadol and propoxyphene is formulated as a sublingual dosage form.

8. The method of claim 1, wherein the combined formulation of tramadol and propoxyphene is formulated as an intranasal dosage form.
